(19) United States
(12) Plant Patent Application Publication
(10) Pub. No.: US 2010/0218296 P1
(43) Pub. Date: Aug. 26, 2010

(54) Sedum plant named 'Zi Juan'

(50) ;

(75) Inventor: Yang Yu Yong, Cheng Gong County (CN)

Correspondence Address: BUCHANAN, INGERSOLL & ROONEY PC, POST OFFICE BOX 1404, ALEXANDRIA, VA, 22313-1404, US

(73) Assignee: SELECTION NEW PLANT S.A.R.L., LeLuc en Provence (FR)

(21) Appl. No.: 12/379,446

(22) Filed: Feb. 23, 2009

(51) Int. Cl. A01H 5/00

(52) U.S. Cl. PLT/479

(57) ABSTRACT

A new and distinct Sedum spectabile plant is provided that originated as a chance sport of the ‘Stardust’ cultivar (non-patented in the United States) while growing in a cultivated state. The growth habit is upright and vigorous. The overall plant stature is medium. Attractive light-green to lime-green foliage is formed that contracts nicely with the light pink blossom coloration. The new cultivar is well suited for providing attractive ornamentation in the gardens.

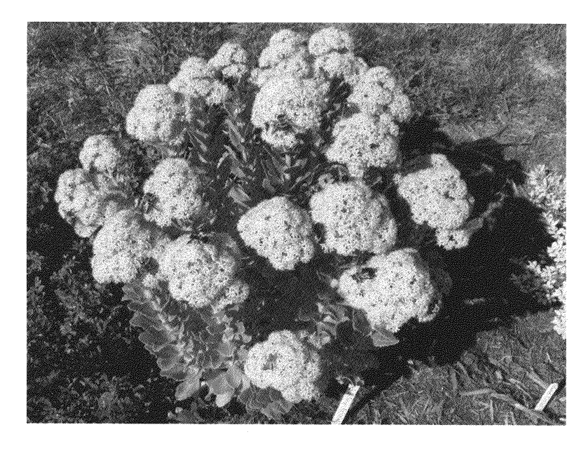

### BOTANICAL/COMMERCIAL CLASSIFICATION

Sedum spectabile/Showy Sedum Plant

### VARIETAL DENOMINATION

cv. Zi Juan

### SUMMARY OF THE INVENTION

A new and distinct cultivar of Sedum plant is provided that is botanically known as Sedum spectabile. Plants of this species are members of the Crassulaceae family.

The new cultivar is a sport of unknown causation of the ‘Stardust’ cultivar (non-patented in the United States) which was discovered while growing in a cultivated area among plants of the ‘Stardust’ cultivar. Had the single plant of the new cultivar not been discovered and preserved it would have been lost to mankind.

It was found that the new Sedum plant exhibits the following combination of characteristics:
(a) forms a profusion of attractive light pink blossoms,
(b) displays an upright vigorous growth habit,
(c) displays a medium overall plant stature,
(d) forms attractive light-green to lime-green foliage that contrasts nicely with the light pink blossom coloration, and
(e) is well suited for providing attractive ornamentation when grown in gardens.

The attractive light pink blossoms of the new cultivar are well borne over the entire plant. The new cultivar well meets the needs of horticultural industry. It can be grown outdoors to advantage in the landscape.

The new cultivar of the present invention can be readily distinguished from the parental ‘Stardust’ cultivar. More specifically, the blossoms of the new cultivar are light pink in coloration unlike the white blossoms of the ‘Stardust’ cultivar. Also, the foliage of the new cultivar displays a lighter green coloration.

Also, the new cultivar can be well distinguished from the ‘Mr. Goodbud’ cultivar (U.S. Plant Pat. No. 17,671). The blossoms of the ‘Mr. Goodbud’ cultivar are red-purple to mauve in coloration unlike the light pink blossoms of the new cultivar. Also, the new cultivar tends to be taller in stature.

The new cultivar has been asexually reproduced at West Grove, Pa., U.S.A., by the use of softwood cuttings. The characteristics of the new cultivar have been demonstrated to be firmly fixed and the new cultivar has been demonstrated to reproduce in a true-to-type manner in subsequence generations.

The new cultivar has been named ‘Zi Juan’, and will be marketed under the PINK SUNDAE trademark.

### BRIEF DESCRIPTION OF THE PHOTOGRAPH

The accompanying photograph shows a specimen of the new cultivar at an age of approximately one year while growing outdoors during September, 2007, at West Grove, Pa., U.S.A.

### DETAILED DESCRIPTION

The following description is based on the observation of the new cultivar at an age of approximately one and one-half years during September while growing outdoors in full sun at West Grove, Pa., U.S.A. Such plant had been asexually reproduced through the use of softwood cuttings. Reference to the R.H.S. Colour Chart of The Royal Horticultural Society, London, England, is provided. Common color terms are accorded their customary dictionary significance.
Origin: Sport of unknown causation of the ‘Stardust’ cultivar of Sedum spectabile.
Type: Herbaceous perennial.
Plant:
   Habit.—upright.
   Stature.—medium.
   Height.—approximately 60 cm on average.
   Width.—approximately 60 cm on average.
   Vigor.—excellent.
   General appearance.—attractive compact plant with showy blossoms.
Branches:
   Stem length.—commonly approximately 60 cm.
   Stem diameter.—approximately 1 cm.
   Color.—Yellow-Green Group 144A.
   Texture.—succulent and glaucous.
Foliage:
   Leaf type.—simple.
   Arrangement.—alternate.
   Configuration.—elliptic.
   Venation.—pinnate.
   Margins.—coarsely and irregularly serrate.
   Apex.—broadly acute.
   Base.—cuneate.
   Blade length.—approximately 6 cm on average.
   Blade width.—approximately 3 cm on average.
   Texture.—glaucous, relatively thick, spongy, and succulent.
   Color.—upper surface: commonly Yellow-Green Group 144A, with the coloration being somewhat variable depending upon the light and stress conditions that are encountered, and with lighter green coloration being displayed under full sun and low water growing conditions. — under surface: commonly between Yellow-Group 144B and Greyed-Green Group 191B.
   Petiole.—the leaves are borne in a sessile manner.
Inflorescence:
   Type.—complete, actinomorphic, stellate, terminal, corymbose, and umbel-like.
   Branching.—commonly with up to approximately 6 main flowering branches per stem.
   Peduncle.—succulent and glaucous.
   Time of blooming.—commonly from September through October at West Grove, Pa., U.S.A.
   Buds.—commonly approximately 5 mm in length on average, and approximately 2.5 mm in width on average.
   Size.—a fully open flower commonly measures approximately 7 mm in depth on average and approximately 10 mm in diameter on average.
   Shape.—ovoid.
   Petal count.—five.
   Petal color.—commonly between Red Group 49C and 49D.
   Petal shape.—lorate and reflexed.
   Petal size.—commonly approximately 4 mm in length on average, and approximately 1.5 mm in width on average.
   Calyx size.—commonly approximately 3 mm in length on average, approximately 1 mm in width on average, campanulate, and commonly divided close to the base.
   Calyx color.—Yellow-Green Group 149B.
   Sepal number.—possesses five lobes.
   Carpels.—prominent, five in number, and separate.
   Pistil size.—commonly approximately 4 mm in length on average, and approximately 1 mm in width on average.
   Pistil color.—Greyed-Purple Group 186B.
   Stamen number.—commonly 10.
   Stamen color.—Greyed-Purple Group 186C.
   Fragrance.—none.
   Lastingness of flowers.—commonly each corymb lasts approximately 5 weeks on average, depending upon the environmental conditions that are encountered.
   Fruit type.—ventrally dehiscent follicle, erect.
The new cultivar can be grown in U.S.D.A. Hardiness Zone Nos. 4 to 9. No unusual susceptibility to diseases and pests has been noted for the new cultivar during observations to date. Root rot may be observed under particularly wet growing conditions. As with other Sedum cultivars, the new cultivar may be damaged by mealy bugs, scale insects, slugs, and snails. Plants of the new ‘Zi Juan’ cultivar have not been observed under all possible environmental conditions to date. Accordingly, it is possible that the phenotypic expression may vary somewhat with changes in light intensity and duration, cultural practices, and other environmental conditions.

I claim:

1. A new and distinct Sedum plant characterized by the following characteristics:
(a) forms a profusion of attractive light pink blossoms,
(b) displays an upright vigorous growth habit,
(c) displays a medium overall stature,
(d) forms attractive light-green to lime-green foliage that contrasts nicely with the light pink blossom coloration, and
(e) is well suited for providing attractive ornamentation when grown in gardens;
substantially as illustrated and described.
